(19) United States
(12) Patent Application Publication
(10) Pub. No.: US 2007/0282456 A1
(43) Pub. Date: Dec. 6, 2007

(54) Compositions and Methods for Myogenesis of Fat-Derived Stem Cells Expressing Telomerase and Myocardin

(75) Inventors: Yong-Jian Geng, Houston, TX (US); Rosalinda Madonna, Houston, TX (US); James T. Willerson, Houston, TX (US)

Correspondence Address: CONLEY ROSE, P.C.;David A. Rose, P. O. BOX 3267, HOUSTON, TX, 77253-3267, US

(73) Assignee: Board of regents of the university of texas system, Austin, TX (US)

(21) Appl. No.: 11/571,985

(22) PCT Filed: Jul. 13, 2005
(86) PCT No.: PCT/US05/24784

§ 371 Date: Jul. 10, 2007

Related U.S. Application Data
(60) Provisional application No. 60/587,360, filed on Jul. 13, 2004.

(51) Int. Cl. A61F 2/02; A61K 35/12; A61P 9/00; C12N 15/63; C12N 5/00; C12Q 1/02

(52) U.S. Cl. 623/23.76; 424/93.21; 435/29; 435/325; 435/455

(57) ABSTRACT

An in vitro method of producing stem cells with potential to develop into cardiovascular myocytes is disclosed which comprises culturing myogenic stem cells obtained from the stromal or mesenchymal compartment of adult adipose tissue in a medium that favors myogenic development of the cells. These myogenic stem cells highly express telomerase and myocardin. A composition comprising fat-derived myogenic stem cells and/or differentiated cardiovascular myocytes and a method for treating a mammalian subject suffering from a cardiovascular disorder are also disclosed.

### CROSS-REFERENCE TO RELATED APPLICATIONS

This application is a National Phase entry under 35 U.S.C. §371 of PCT Application Number PCT/US2005/024784 filed Jul. 13, 2005, which claims the priority of U. S. Provisional Application No. 60/587,360 filed Jul. 13, 2004, both of which are hereby incorporated herein by reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant Nos. R01HL59249 and R01HL69509S awarded by the National Institutes of Health.

### BACKGROUND OF THE INVENTION

1. Field of the Invention

The present invention generally relates to the in vitro production of cardiovascular myocytes, and more particularly to methods for producing differentiated cardiovascular myocytes from fat-derived myogenic stem cells. The invention also pertains to compositions and methods for treating an individual suffering from a cardiovascular disorder by administering such fat-derived myogenic stem cells and/or differentiated cardiovascular myocytes.

2. Description of Related Art

Stem cell transplantation is emerging as a potentially novel therapy for patients with heart failure or myocardial infarction. Several research teams, including our own, have been pursuing clinical trials for treating heart failure patients with adult stem cells derived from their own bone marrow (1) and skeletal muscle satellites (2). Recent studies provide compelling evidence that pluripotent stem cells exist in adult adipose tissue, and they may be capable of differentiating into a variety of cell lineages including those in cardiovascular tissues in addition to adipocytes (3, 4). However, biological characteristics of adult stem cells residing in the adipose tissue are unclear. Thus far, the majority of multi- or oligopotent stem cells have been found in the stromal compartment of the adipose tissue referred to as mesenchymal stem cells (MSC). Expression of markers for undifferentiated stem cells has been reported in some of the adipose stromal cells (3). Embryologically, the adipose tissue stromal or mesenchymal cells and cardiovascular cells develop from the mesoderm layers. Very recently, several investigations on adult stromal cells freshly isolated from both animal and human adipose tissue have shown that in culture, adult adipose tissue-derived adult stem cells can be induced to differentiate or transdifferentiate into cardiac myogenic cells (5-7). The molecular mechanism governing the potential for adult stem cells to differentiate into mature cardiac and/or vascular myocytes remains obscure. Two molecules, telomerase and myocardin, expressed by stem cells may play regulatory roles in myogenic stem cell development.

Telomerase (8), a ribonucleoprotein complex, catalyses addition of the oligonucleotide (TTAGGG) repeats onto the repetitive DNA structure, telomeres, at the ends of linear chromosomes. The telomerase-catalyzed DNA addition prevents telomere shortening and stabilizes chromosomes (9). Telomerase contains the RNA-dependent DNA polymerase (reverse transcriptase) activity with its RNA component (complementary to the telomeric single stranded overhang) as a template in order to synthesize the TTAGGG repeats directly onto telomeric ends. This extension of the 3′ DNA template permits additional replication of the 5′ end of the lagging strand, thus compensating for the telomere shortening that occur in its absence. Telomerase exists abundantly in embryonic stem cells (ESC) and in adult germline cells, but is almost undetectable in mature somatic cells except for actively proliferating cells of renewal tissues (10). The telomerase-mediated maintenance of telomere length contributes to pluripotency or “stemness” of cellular lineage differentiation in mammalian tissues. In the heart, telomerase activity is associated with myogenic cell survival, growth, and differentiation (11-14). Altered expression of telomerase also occurs during the development of heart failure (15).

Myocardin, a transcriptional coactivator of serum response factor (SRF), has been recently identified as a key regulator of myogenesis during the development of the heart (16, 17) as well as blood vessels (18-20). This cardiac and vascular muscle-specific transcriptional regulator is critical for cardiovascular myocyte development. It may interact or be regulated by other transcriptional factors such as the myocardin-related transcription factors (MRTFs) (21), and Nkx2.5 or Csx (17), an evolutionarily conserved cardiac transcription factor of the homeobox gene family.

It has been debated among investigators as to whether multipotent stem cells exist in adult somatic tissues and whether, when transplanted into other types of tissues or organs, the adult stem cells can differentiate into functionally specialized cells for the host tissues. In essence, adult stem cells should share the same or similar biological characteristics with embryonic stem cells, i.e., expression of cellular proteins important for maintaining their “stemness” or pluripotency.

### SUMMARY OF THE INVENTION

The present invention provides methods and compositions for generating or repairing cardiovascular tissue using myogenic stem cells obtained from adipose tissue. It is proposed herein that adult adipose tissue will serve as an alternative source of stem cells for cardiac cellular therapy. As an alternative stem cell reservoir, adipose tissue has several advantages over other sources of stem cells for cellular therapy. Fat deposits in the body are abundant, accessible and replenishable. Adult stem cells can be isolated from liposuction waste tissue by collagenase digestion and differential centrifugation.

In accordance with certain embodiments of the present invention, a method of treating a mammalian subject suffering from a cardiovascular disorder is provided. The method generally comprises (a) providing a plurality of myogenic stem cells obtained from the mesenchymal compartment of adult adipose tissue; (b) causing the stem cells to proliferate; (c) inducing differentiation of said stem cells into functional mature cardiovascular myocytes; (d) implanting at a cardiovascular site in said subject a composition comprising said stem cells and/or said mature cardiovascular myocytes. In some embodiments, the method comprises providing muscle cells in the heart of the subject. In some embodiments, the method comprises providing muscle cells in a blood vessel of the subject. Some embodiments of the method include proliferating and isolating myogenic stem cells in vitro prior to implantation in the subject.

In preferred embodiments, the myogenic stem cells are capable of producing telomerase and myocardin when implanted at the cardiovascular site. In some embodiments, the method comprises genetically engineering the stem cells to co-express telomerase and myocardin. Stem cell senescence and apoptosis is preferably deterred or prevented by such telomerase production. Differentiation of the MSCs is also preferably promoted by the produced telomerase and myocardin. In some embodiments, the method includes growing the myogenic stem cells in vitro prior to implantation. In some embodiments, the method includes repopulating cardiac cells at the implantation site, to ameliorate chronic heart failure, for instance. In some embodiments damaged myocardium, such as a myocardial infarction, is repaired by implanting the cultured cells. In still other embodiments, the method of treating a mammalian subject suffering from a cardiovascular disorder includes implanting the stem cells and/or differentiated cardiovascular myocytes at the site of a vascular defect, to repopulate the site with vascular cells.

In some embodiments, an above-described method also includes (a) liposuctioning adipose tissue from the stromal or mesenchymal compartment of the body of said subject or from that of a donor, to provide a quantity of removed adipose tissue; (b) enzymatically digesting proteins and DNA in said removed adipose tissue, to provide a quantity of digested adipose tissue; and (c) separating live adipocytes from other cells in said digested tissue. The order in which any method steps are recited herein is not intended to imply a fixed order in which the various steps must be carried out, unless so stated.

In some embodiments, an above-described method of treatment comprises identifying, selecting and growing cardiovascular stem cells in vitro. In some embodiments, an above-described method comprises identifying, culturing and selecting cardiac myogenic cells said heart muscle cell progenitors, or both of those. In some embodiments vascular myogenic cells, vascular smooth muscle cell progenitors, or a combination of those, are identified, cultured and selected. In some embodiments, the method includes identifying, culturing and selecting endothelial cell progenitor cells. The above-mentioned selecting steps preferably employ a cell sorting technique as is known in the art. In some embodiments, the treatment method includes isolating and transplanting stem cells with the potential to differentiate into smooth muscle cells. In some embodiments, the treatment method also includes isolating and transplanting endothelial cells.

Also provided in accordance with certain embodiments of the present invention is an in vitro method of producing cardiovascular myocytes. This in vitro method comprises (a) isolating myogenic stem cells from the mesenchymal compartment of adult adipose tissue, (b) culturing those cells in a medium that favors myogenic development of the cells, and then (c) harvesting the resulting cardiovascular myocytes from the culture medium. In some embodiments step (a) includes transfecting the resulting isolated myogenic stem cells with cDNA encoding telomerase and myocardin, and step (b) includes (b-1) culturing the resulting transfected cells in a medium that favors myogenic development of said stem cells; (b-2) expressing the transfected telomerase and myocardin cDNA; and (b-3) expressing at least one other gene in said transfected myogenic stem cells encoding at least one protein associated with telomerase and myocardin function.

In some embodiments, the method comprises (a) plating stromal or mesenchymal cells at a density of about 10,000 cells/cm² in an initial cell culture medium comprising DMEM:F12 medium supplemented with penicillin (100 U/mL), streptomycin sulfate (100 μg/mL) and 10% fetal bovine serum (FBS); (b) replacing said initial medium with an inducing medium comprising Iscove's MDM liquid medium (Gibco, Carlsbad Calif.) supplemented with L-glutamine (2 mmol/L), penicillin (100 U/mL), streptomycin sulfate (100 μg/mL), 0.1 mM nonessential amino acid, 10⁻⁴ mol/L 2-mercaptoethanol and 20% FBS, to induce cardiomyogenesis; and (c) allowing cardiovascular myocytes to grow until confluence is reached. In some embodiments, the method includes testing a sample of cells for production of telomerase, myocardin, or for one or more cardiomyogenic protein. In some embodiments all or some combinations of those tests are performed.

In accordance with still another embodiment of the present invention, a composition is provided for treating a cardiovascular disorder such as a myocardial infarction, chronic heart failure, atherosclerosis, hypertension, or a site of vascular disease or damage. The composition comprises a plurality of cardiomyocytes prepared as described above, and a pharmaceutically acceptable carrier. Suitable carriers as are used with conventional implantable cellular compositions are known in the art. These and other embodiments, features and advantages of the present invention will become apparent with reference to the following description and drawings.

### BRIEF DESCRIPTION OF THE DRAWINGS

FIG. 1. Immunofluorescence and reverse transcription-polymerase chain reaction (RT-PCR) for telomerase-reverse transcriptase (TERT) in adipose tissue-derived mesenchymal stem cells. Immunofluorescence assay was performed with rabbit polyclonal antibodies to the telomerase catalytic subunit TERT in primarily cultured murine (a-c) and porcine (d-f) MSCs. FITC-conjugated anti-rabbit IgG was used as the secondary antibody. Nuclear counterstaining was conducted with the fluorochrome DAPI. a and d, TERT immunofluorescence; b and e, DAPI nuclear staining in the same field of a and d, respectively; c and f, merged images of a+b and d+f. Arrows indicate green immunofluorescence of TERT in the nuclei stained with DAPI emitting blue fluorescence. Images were taken using x40 objective. g, RT-PCR performed with total RNA isolated from murine mesenchymal stem cells (mMSC) and embryonic stem cells (mESC). f, RT-PCR with total RNA from mMSC (lane 2), dog MSC (dMSC) (lane 3), Mesc (lane 4), pig MSC (pMSC) (lane 5), murine smooth muscle cells (mSMC) (lane 6), human SMC (hSMC) (lane 7) and murine endothelial cells (mEC) (lane 8).

FIG. 2. Immunoblotting for TERT and β-actin in adipose tissue-derived mesenchymal stem cells. Total proteins were extracted from MSCs of mice (mMSC) (lane 7), dogs (dMSC) (lane 5), pigs (pMSC) (lane 6) as well as from human endothelial cells (hEC) (lane 1), human smooth muscle cells (hSMC) (lane 2), murine smooth muscle cells (mSMC) (lane 3) and murine embryonic stem cells (mESC) (lane 4), separated by SDS-PAGE, electrotransferred onto nitrocellulose membranes, and then stained with a polyclonal rabbit anti-TERT antibody which cross-reacts with the murine, human, pig and dog TERT antigens (a). The same membrane was reprobed with antibody to β-actin (c). Semiquantification of TERT and β-actin was conducted by densitometry (b and d, respectively). Results are representative of three separate experiments.

FIG. 3. TRAP assays for telomerase activity in adipose tissue-derived MSCs, human and murine vascular cells, and Hela cells. MSCs derived from the adipose tissue of mice (mMSC) as well as from cultured murine aortic endothelial cells (mEC), human coronary smooth muscle cells (hSMC), and Hela cells, were prepared for assessing the telomerase activity by using the telomeric repeat amplification protocol (TRAP). Standard curve of telomerase activity was generated using serial dilution of TSR8 control templates. a, Representative TRAP gel image showing typical ladders of PCR-amplified telomeric repeats. b, Fluorometry of the telomerase activity in three experiments.

FIG. 4. RT-PCR for myocardin-A mRNA expression in murine MSCs, embryoid bodies (EB) and aorta tissue as well as mature vascular cells. RT-PCR was performed with total RNA from differentiated murine embryoid bodies (mEB) at day 10 (lane 2), murine mesenchymal stem cells (mMSC) after 1 week culture (lane 3), differentiated murine embryoid bodies (mEB) at 14 days from plating (lane 4), and mMSC after 3 week culture (lane 4), murine aorta (lane 5). Templates were omitted in the reaction as the negative control (lane 8). Expected size of PCR products from transcripts of myocardin A (a) and G3PDH “house-keeping” control (b) are 575 bp and 475 bp, respectively. PCR product bands were quantified by densitometry (c). Data represents means±S.D. of three experiments.

FIG. 5. Immunoblotting for myocardin and TERT in non-differentiated and differentiated MSCs as well as embryoid bodies with or without contractile myocytes. Nuclear proteins extracted from murine ESC, aortic SMC and adipose tissue-derived MSC (panel a) or total proteins from pig coronary artery (pLAD), differentiated murine MSC (mMSC), murine embryoid bodies (mEB) and adult mouse heart were separated by SDS-PAGE, electrotransferred to membranes, and probed with anti-myocardin and anti-TERT antibodies. Control blotting was performed with preimmune serum.

FIG. 6. Immunofluorescence and immunoblotting for expression of cardiomyogenic markers in the colonies of adipose tissue-derived MSCs containing contractile myogenic cells. Panels a−c: Immunofluorescent microscopy of differentiated murine adipose tissue-derived MSCs. Nuclear counterstaining with DAPI yields blue fluorescence in nuclei. a, anti-cardiac α-myosin; b, anti-ryanodine receptor; and c, anti-α-sarcomeric actinin. Panel d: Digital video image (panel d and supplemental Figure online) of the MSC colonies with spontaneously beating cells 14 days after culture initiation was taken by inverted fluorescence microscopy with a time-lapse digital camera. Panels e and f: Immunoblotting for α-sarcomeric actinin and β-actin in contractile and non-contractile differentiated MSCs. The intensity of protein bands was determined by densitometry. e, immunostained bands for cardiac sarcomeric α-actinin (upper panel) and for β-actin (lower panel); and b, quantitation of band intensity by densitometry. Data represent means±S.D. of three separate experiments.

FIG. 7. Immunoblotting for expression of smooth muscle α-actin in contractile and non-contractile murine MSCs and embryoid bodies (mEBs) as well as in mature vascular cells. Immunoblotting with antibodies against smooth muscle α-actin and β-actin in murine MSC colonies (mMSC) and mEBs with or without contractile and non-contractile cells as well as in H9c2 myoblasts and murine smooth muscle cells (mSMC) and endothelial cells (mEC). Upper panel: anti-smooth muscle α-actin; and lower panel, anti-β-actin.

### DETAILED DESCRIPTION OF THE PREFERRED EMBODIMENTS

Overview

We discovered that the stromal compartment of adult adipose tissue contains myogenic stem cells highly expressing telomerase and myocardin. Highly expressed in embryonic stem cells (ESCs), telomerase acts as a reverse transcriptase that maintains nuclear telomere length and replication potential, while myocardin, a transcriptional co-activator of serum response factor, controls cardiovascular myogenesis. Examination of the telomerase catalytic subunit or telomerase-reverse transcriptase (TERT) mRNA and protein revealed higher levels of telomerase expression in mesenchymal stem cells (MSCs) isolated from the adipose tissue of adult animals (e.g., mice, dogs and pigs). In contrast, TERT expression was not appreciable in mature, resting cardiovascular cells. The telemetric repeat amplification protocol (TRAP) assay for telomerase activity further demonstrated the presence of biologically active telomerase in the adipose tissue-derived MSCs at levels comparable to that in ESCs. Telomerase-positive MSCs also produced significant quantities of mRNA and protein of the promyogenic transcription cofactor myocardin-A. Similar to differentiating ESCs in embryoid bodies (EBs), the MSCs with dual expression of telomerase and myocardin developed various colonies, and some of them contain contractile myogenic cells after 2-3 weeks in culture. The spontaneously contracting myocytes emerged in a synchronized fashion with a rhythm of about 100 beats per min, and the visible myocyte contraction lasted at least for two weeks. The contractile but not non-contractile colonies exhibited stronger immunoreactivity towards cardiac and vascular myogenic markers, e.g., cardiac α-sarcomeric actinin and smooth muscle α-actin. Thus, the stromal or mesenchymal compartment of adult adipose tissue contains cardiovascular myogenic stem cells with biologically active telomerase and the myogenic transcription cofactor myocardin A. These results, which are described in more detail below, suggest that adult adipose tissue may serve as an alternative resource of stem cells for cardiac cellular therapy.

We hypothesized that in a cardiac and vascular muscle-specific myogenic stem cell, at least two groups of genes contribute to the potential of survival, growth and differentiation in cardiovascular myogenic stem cells: the first group consists of genes (e.g., telomerase) that support long-term survival and prevent senescence or apoptosis; and the second group includes genes (e.g., myocardin) that regulate myogenic differentiation in response to intrinsic or extrinsic factors. Accordingly, studies were designed to seek evidence that the stromal or mesenchymal compartment of adult adipose tissue contains telomerase- and myocardin-positive myogenic stem cells capable of differentiating into functional mature cardiovascular myocytes. The data from the current study indicate the existence of pluripotent myogenic stem cells in the mesenchymal compartment of adult adipose tissue, highly expressing bioactive telomerase and the promyogenic protein myocardin and capable of differentiating into cardiac as well as vascular myocytes in culture. Thus, the adult adipose tissue may serve as a potential source of myogenic stem cells for cardiovascular cellular therapy.

Methods and Materials

Cell isolation and culture. We isolated and cultured mesenchymal cells from the adipose tissue of adult animals, including mice, dogs and pigs, following collagenase digestion. In brief, abdominal adipose tissue was resected, minced, and digested with type II collagenase (Worthington Biochemical Co., Lakewood, N.J.) at 37° C. for 30 min. After adipocytes were removed from top layers following centrifugation, stromal or mesenchymal cell populations were collected and plated (10,000 cells/cm² density) in DMEM:F12 medium supplemented with penicillin (100 U/mL), streptomycin sulfate (100 μg/mL) and 10% fetal bovine serum (FBS). Cardiomyogenesis was induced in Iscove's MDM liquid medium (Gibco, Carlsbad, Calif.) supplemented with L-glutamine (2 mmol/L), penicillin (100 U/mL), streptomycin sulfate (100 μg/mL), 0.1 mM nonessential amino acid, 10⁻⁴ mol/L 2-mercaptoethanol and 20% FBS. Cells were allowed to grow for 15 days until confluence. Growth pattern and morphology were closely monitored under a phase-contrast microscope. As positive and negative controls for adipose tissue-derived stem cells, the following cell lineages were cultured: murine aortic endothelial cells and smooth muscle cells; human coronary smooth muscle cells; human HeLa cells; rat H9c2 myoblasts (American Type Culture Collection, Manassas, Va.); and murine CCE embryonic stem cells (ESC) (StemCell Technologies, Vancouver, BC Canada).

Immunofluorescence and Immunoblotting. Cells grown in 8-well glass chamber slides (LabTek, Nalge/Nunc) were fixed with 4% paraformaldehyde, permeabilized and then blocked in PBS containing 1% BSA for 30 min. Cells were incubated for 1 h at 4° C. in PBS plus 1% BSA with primary antibodies against following antigens: (1) cardiac sarcomeric α-actinin (Sigma-Aldrich, St. Louis, Mo.); (2) the ryanodine receptor (Santa Cruz Biotechnologies, Santa Cruz, Calif.); (3) TERT (Santa Cruz Biotechnologies, Santa Cruz, Calif.), and (4) cardiac α-myosin (Sigma); and (4) smooth muscle α-actin (Sigma). Polyclonal rabbit anti-myocardin A antibodies were prepared in our lab with synthetic myocardin peptides. After incubation with primary antibodies, cells were washed in PBS and incubated for 30 min with Texas Red- or FITC-conjugated anti-goat or rabbit secondary antibodies. The slides were washed and mounted with a solution containing 4′-6-Diamidino-2-phenylindole (DAPI) to detect nuclei (VectaShield, Vector Labs, Burlingame, Calif.). After staining, samples were viewed with an inverted fluorescent microscope (Olympus) or with a confocal scanning fluorescent microscope (Olympus). For Immunoblotting, total proteins were isolated in ice-cold RIPA buffer, separated under reducing conditions and electro-blotted to PVDF (polyvinylidene fluoride) membranes (Immobilon-P, Millipore Bedford, Mass.). After blocking, the membranes were incubated overnight at 4° C. with the following primary antibodies to cardiac and smooth muscle markers. The blots were incubated with horseradish peroxidase-coupled secondary antibodies, washed and developed by using a SuperSignal West Pico Chemiluminescent Substrate Kit (Pierce, Rockford, Ill.). The intensity of each immunoreactive protein band was quantified by densitometry.

TRAP assay. Telomerase activity was quantified using TRAPeze Telomerase Detection Kit (Intergen Chemicon, Temecula, Calif.), according to the manufacturer's protocol. Briefly, 1×10⁶ cells per sample were lysed in 200 μl of ice-cold 1× Chaps lysis buffer (0,5% 3-[(3-cholamidopropyl)dimethylammonio]-1-propanesulfonate, 10 mmol/L Tris-HCL (pH 7.4), 1 mmol/L MgCl2, 1 mmol/L EGTA, 10% glycerol, 5 mmol/L β-mercaptoethanol). After 30 min incubation on ice, the lysate was centrifuged at 12,000 g for 20 min at 4° C., and the supernatant assayed for protein concentration using the Bradford method (BioRad Laboratories, Hercules, Calif.). Cell lysates were titrated ranging from 0.5 to 3.5 μg protein per assay. The telomeric repeat amplification protocol (TRAP) reaction was performed using 2 μl of protein supernatant, 10 μl 5× TRAPeze XL reaction mix (100 mM Tris-Hcl pH 8.3, 7.5 mM MgCl2, 315 mM KCl, 0.25% Tween 20, 5 mM EGTA, 0.5 mg/mL BSA, TS primer, RP Amplifuor primer, K2 Amplifuor primer, TSK2 template, dA, dC, dG and dTTP), 0.4 μl Taq Polymerase (5 units/l) and water to a final volume of 50 μl. Telomere extension was performed at 30° C. for 30 min, followed by 3-step PCR at 94° C./30 sec, 59° C./30 sec, 72° C./i min for 36 cycles. The final extension step was performed at 72° C. for 3 min. A standard curve of telomerase activity was generated using serial dilution of TSR8 control template. TSR8 is an oligonucleotide with a sequence identical to the TS primer extended with 8 telomeric repeats AG(GGTTAG)₇. This standard curve permits the calculation of the amount of TS primers with telomeric repeats extended by telomerase in a given extract. PCR products were separated in a non-denaturing 12% PAGE in 0.5×TBE at 5 V/cm. The gel was stained using Sybr green and was photographed. Using the ImageQuant software (Kodak, Hercules, Calif.), we quantified the signal intensity by determining the densitometry of each repeat ladder corrected for the background and expressing the activity as total product generated. We also quantified the activities by fluorescence measurement in 96 well plates, after setting the excitation/emission parameters for fluoresceine (495/516 nm) and sulforodamine (600/620 nm), using a fluorescence plate reader.

Reverse transcription-polymerase chain reaction (RT-PCR). Total RNA (1 μg/reaction) isolated from adipose mesenchymal stem cells with TRIzol reagents (Invitrogen) was reverse-transcribed into cDNA. The telomerase and myocardin cDNA templates were amplified for polymerase chain reaction with Taq DNA polymerase and specific primers, respectively for murine telomerase (forward primer, 5′-TGTCATCCCTGAAAGAGCTG-3′ and reverse primer, 5′-GTCTGGTCTCAATAAATGGC-3′) and myocardin (forward primer, 5′-TGGATAGTGCCAAGACTGAA-3′ and reverse primer, 5′-ACAGCAGTGTGCACAGGAAT-3′). The reaction was optimized and run under conditions of linearity with respect to input RNA.

Statistical Analyses. Two-group comparisons were performed by the Student's t-test for unpaired values. Comparisons in differences between multiple groups were performed by Analysis of Variance (ANOVA), and the existence of individual differences, in case of significant F values at ANOVA, tested by Scheffé's multiple contrasts. Significance was established when p values were less than 0.05.

Results

Adipose Tissue-Derived Mesenchymal Stem Cells Express Telomerase.

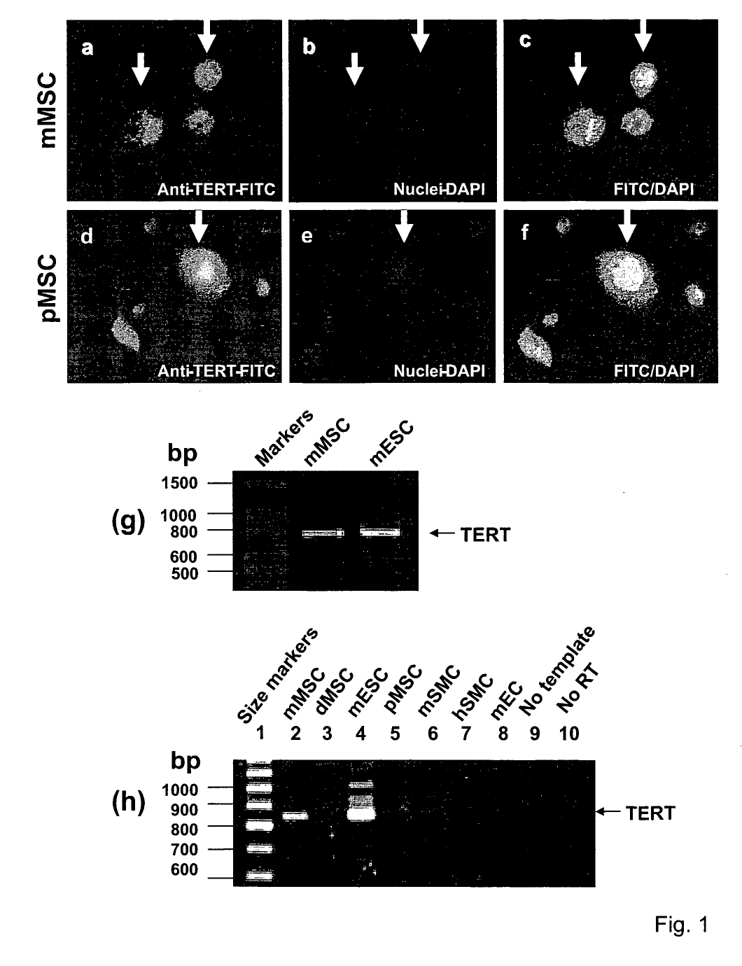

We examined expression of telomerase in MSCs isolated from the stromal compartment of the adipose tissue of adult mice (2-4 months), dogs (1-2 years) and pigs (1-2 years). We detected strong immunofluorescence for the telomerase catalytic subunit TERT in the primary cultures of MSCs from all the animals tested (FIGS. 1a-f). All the TERT-positive MSCs displayed similar patterns of intracellular TERT immunofluorescence. Although both the cytoplasm and nuclei contained immunoreactive TERT, the signals from the nuclei appeared to be more pronounced than those from the cytoplasm, suggesting translocation of TERT into the nucleus from the cytoplasm. In the cultures, few cells showed the morphology of adipocytes, such as accumulation of intracellular lipids as tested by staining with Oil Red O. The TERT positive cells expressed little perillipin, an intracellular lipid-binding protein selectively expressed by adipocytes, suggesting that they were not adipogenic cells.

Analysis of mRNA by RT-PCR further confirmed expression of TERT in the adipose tissue-derived MSCs. Clear signals for TERT mRNA existed at steady-state levels in murine MSCs (FIG. 1g) as well as in canine and porcine MSCs (FIG. 1h). As the positive control, we also examined the TERT mRNA levels in murine embryonic cells. Expectedly, in the RT-PCR assays, we found stronger signals for TERT mRNA in ESCs (FIG. 1h). In contrast, the TERT mRNA signals were almost undetectable or weakly detected in the mature, differentiated smooth muscle cells and endothelial cells from either rodent or human (FIG. 1h).

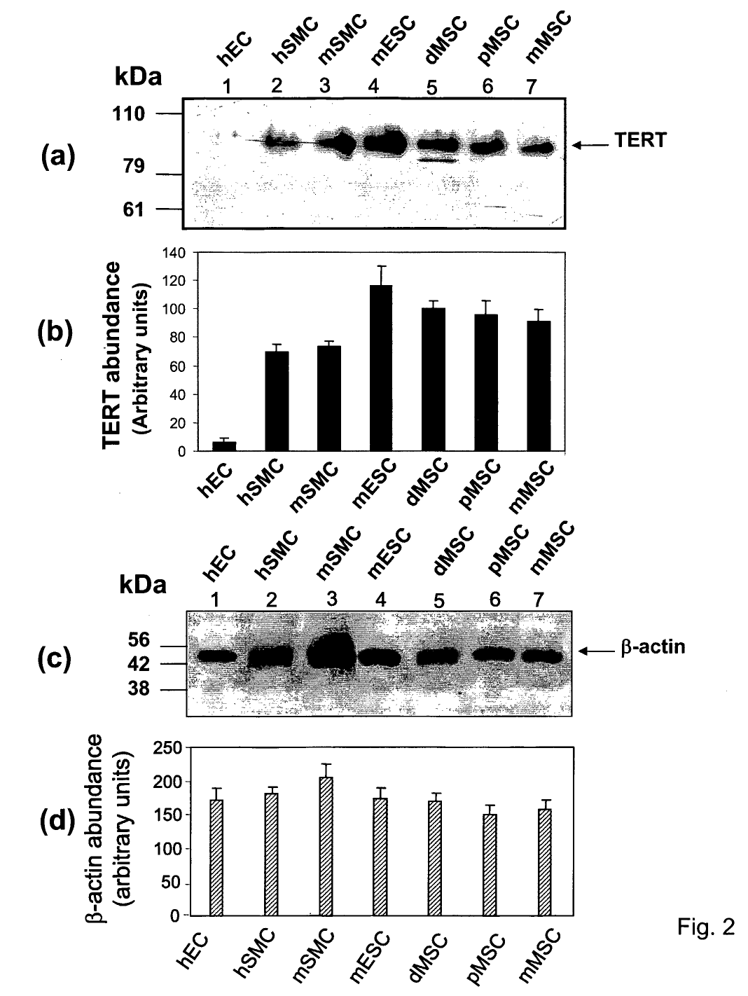

Immunoblotting with the total protein extracts with the same antibodies for immunofluorescence revealed the presence of immunoreactive TERT bands in MSCs of all the species tested, even though the intensity of TERT bands was less pronounced when compared to that in ESCs (FIG. 2). There was no major difference in TERT expression between the mouse, dog and pig MSCs (FIG. 2b). In contrast, mature vascular cells, such as murine smooth muscle cells and human mature endothelial cells, showed little immunoreactive telomerase while they produced almost equal or higher levels of β-actin (FIGS. 2c and d). Thus, the results indicate that similar to ESCs, MSCs derived from adult adipose tissue were expressing the telomerase catalytic subunit TERT.

Telomerase is Biologically Activated in Adipose Tissue-Derived Mesenchymal Stem Cells.

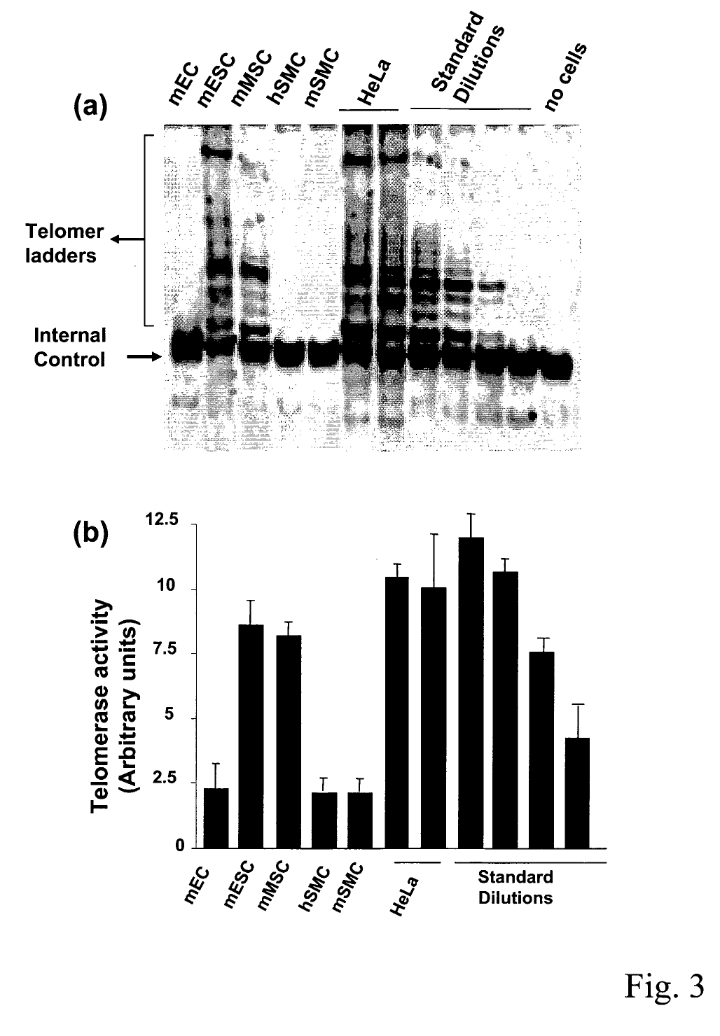

In order to determine the biological activity of telomerase in MSCs, we performed the highly sensitive TRAP assays to determine the telomerase activity in murine ESCs and MSCs as well as mature vascular cells, i.e., endothelial cells and smooth muscle cells. In the TRAP assays, cellular telomerase acts as a reverse-transcriptase that synthesizes DNA fragments at different lengths of telomeric repeats, yielding a pattern of telomeric DNA ladders on the ethidium bromide-stained agarose gels which are visible under UV light. By incubating the MSC lysates with telomeric templates, we clearly observed the formation of DNA ladders indicative of the telomerase-mediated synthesis of the telomere repeats (TTAGGG). The positive signals were strong in the murine ESCs and in the human Hela cells known to exhibit high levels of the telomerase activities (FIG. 3). Interestingly, MSCs isolated from the adipose tissue exhibited the telomerase activity at almost equal levels to those seen in murine ESCs as well as Hela cells cultured under the identical experimental conditions (FIG. 3). In contrast, adult vascular cells expressed unappreciable levels of telomerase activity (FIG. 3). We found no telomerase activity in the reactions without cellular protein extracts, indicating the selectivity of the assays. Consistent with the results from analysis of TERT expression, the data from the TRAP assay indicate that MSCs from adult adipose tissue express biologically active telomerase.

Adipose Tissue-Derived Mesenchymal Stem Cells Express Myocardin-A.

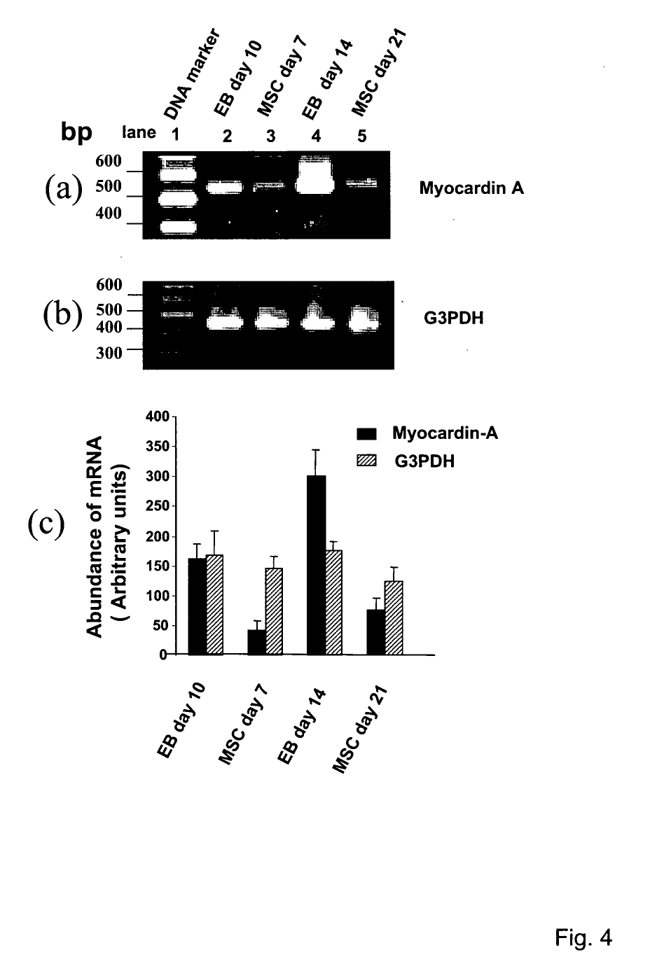

Myocardin has been implicated in regulation of cardiac and vascular muscle-specific myogenic cell differentiation (17, 20, 21). In order to determine whether myocardin exists in the telomerase-expressing MSCs in a pattern similar to that seen in embryonic cells, we performed RT-PCR for myocardin with total RNA isolated from MSCs and murine embryoid bodies (EBs) derived from ESCs. We found that prolonging cultures in MSCs from 7 days to 21 days or in EBs from 10 days to 14 days markedly increased intracellular myocardin mRNA levels (FIG. 4), suggesting that expression of myocardin in MSCs and ESCs might be differentiation-dependent. The myocardin mRNA levels in ESCs were however lower than that in differentiating EBs, while MSCs and EBs showed almost the same levels of the “house-keeping” gene G3PDH.

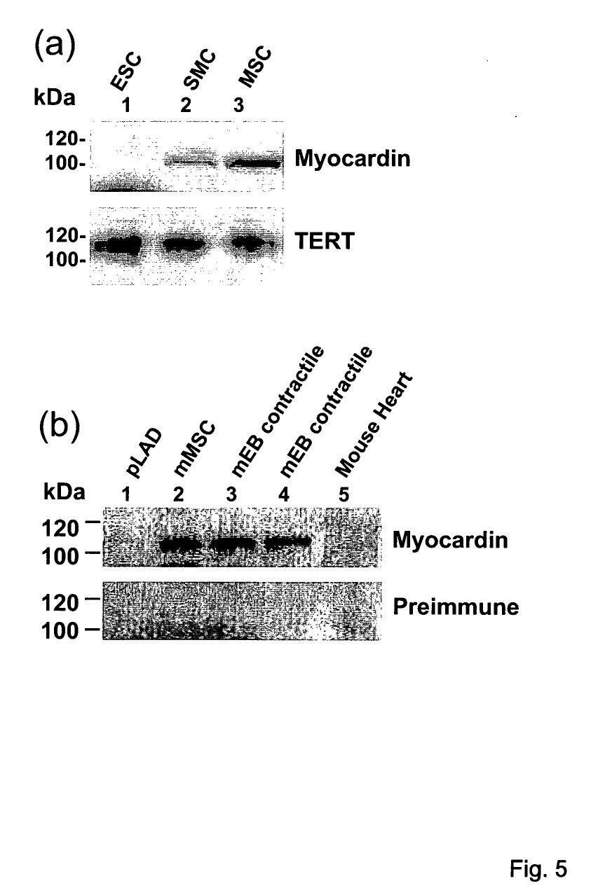

By immunoblotting with anti-myocardin A antibodies, we examined myocardin protein expression in the embryonic and adult stem cells as well as mature cardiovascular cells. We observed a clear protein band at the molecular weight of about 100 kDa in MSCs cultured for 7 days from the stromal compartment of murine adipose tissue (FIG. 5a), consistent with the mRNA expression. Undifferentiated ESCs cultured with leukemia inhibitor factor expressed negligible levels of myocardin-A while they produced TERT significantly (FIG. 5). However, in differentiating EBs, in particular those with contractile myocytes, there appeared abundant myocardin-A. Differentiating MSCs also contained substantial amounts of myocardin-A. The myocardin expression in the adipose tissue-derived MSCs occurred after one week in culture and became greater after 3 weeks in culture, indicating the time-dependence of myocardin gene expression. Vascular smooth muscle cells in culture exhibited positive signals for myocardin, albeit to much weaker degrees compared to MSCs. In contrast, there was not appreciable expression of myocardin-A in the mature, resting smooth muscle and heart muscle (FIG. 5). Thus, resembling differentiating ESCs, the telomerase-positive MSCs from adipose tissue could produce the cardiovascular muscle-specific transcriptional co-activator myocardin-A.

Adipose Tissue-Derived Mesenchymal Stem Cells Differentiate into Contractile Cardiomyocytes.

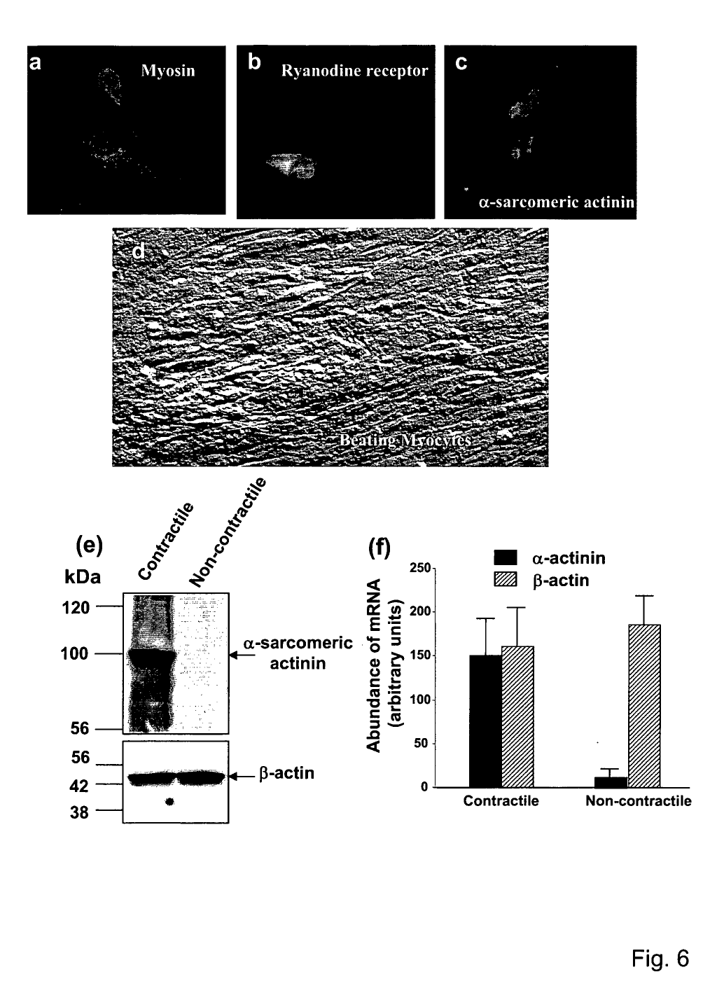

In consideration of the promyogenic role of myocardin during the development of the cardiovascular system (16), we tested whether MSCs positive for both telomerase and myocardin have the differentiational potentials of cardiovascular myogenic cells. We established a cell culture system which favors myogenic development of stem cells. For comparison, we examined different groups of cells for their capability of development into contractile stem cells, including (i) murine MSCs expressing both telomerase and myocardin-A; (ii) undifferentiated murine ESCs with abundant telomerase but little myocardin; and (iii) mature vascular smooth muscle cells with low telomerase but positive for myocardin. Interestingly, we observed that in the cultures for two weeks, MSCs from the adult adipose tissue formed colonies and synthesized cardiac myogenic markers, such as myosin (FIG. 6 a), α-sarcomeric actinin (FIG. 6b), and the ryanodine receptor (FIG. 6c), while they remained positive for TERT and myocardin. Astonishingly, we observed that without any additional stimulation, contractile cells developed spontaneously between days 14-21 in some of the MSC colonies (FIG. 6d). The beating myogenic cells clustered and formed junctions with each other as they contracted rhythmically in a synchronized fashion clearly visible under a microscope. Recorded by a digital video camera (FIG. 6d supplement), the rhythm of the myogenic cell contraction seemed fairly stable and regular with the beating rate (approximately 100 beats/min). The visible cell contraction lasted almost for two weeks, and then gradually weakened. Under the same culture condition, telomerase-positive ESCs did not differentiate into EBs with contractile myocytes. However, after forming EBs in the “hanger-drop” culture system, colonies with contractile myocytes developed with increased expression of myocardin. Cultured vascular smooth muscle cells expressed myocardin-A but little telomerase, and they did not develop into any visible contractile cells in the cultures suggesting that they were not myogenic stem cells.

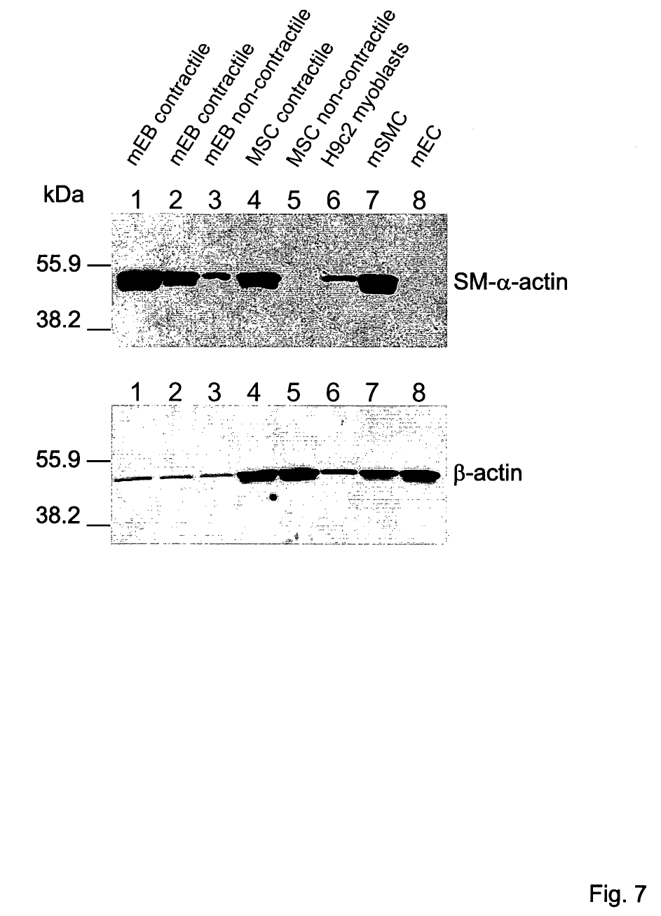

For further characterization of the contractile myogenic cells developed from MSCs, we extracted proteins from the colonies of MSCs with and without beating myogenic cells for analysis of cardiac myogenic markers such as cardiac α-sarcomeric actinin. As expected, we observed that the MSC colonies with beating cells expressed immunoreactive cardiac sarcomeric actinin, whereas those without contractile myogenic cells did not express this cardiac cytoskeletal protein (FIGS. 6e and f). Because myocardin-A has been also shown to regulate smooth muscle differentiation, we performed immunoblotting with antibodies against vascular smooth muscle α-actin in MSCs as well as other control cells. Strikingly, MSCs with contractile myocytes but not those without contractile cells expressed the smooth muscle actin (FIG. 7). Similarly, murine differentiating ESCs or EBs showed a high immunoreactivity with the anti-smooth muscle α-actin antibody. Undifferentiated H9c2 cells produced moderate levels of the smooth muscle actin, while endothelial cells did not express this protein (FIG. 7). In contrast, there was no difference in expression of β-actin between the MSC colonies with and without beating cells. Taken together, the telomerase and myocardin expressing MSCs were capable of replicating themselves and differentiating into contractile cardiac myocytes as well as vascular smooth muscle cells, suggesting that they might function as myogenic stem cells.

Discussion

Progressive loss of mature, functional cardiomyocytes characterizes the failing hearts with infarction or prolonged ischemia. Because of their limited capacity of regeneration, the adult hearts need to recruit external stem cells to repopulate cardiomyocytes and replace damaged or injured ones. Thus far, animal studies (22) have shown that a variety of stem cell or stem cell-like lineages isolated from embryonic and adult tissues can differentiate or transdifferentiate into cardiovascular cells, and thereby may have potential for cardiac cellular therapy. These stem cells include embryonic stem cells (10, 23), fetal myoblasts (24, 25), bone marrow stem cells (26), skeletal satellite myoblasts (27), and endothelial cell progenitors (28). While the studies of embryonic stem cells still remain in the laboratory setting, adult stem cells, mostly from the bone marrow (1) and skeletal muscle (27), have been recently used for treating patients with prior myocardial infarction and chronic heart failure. There has been a major debate concerning the pluripotency or “stemness” of stem cells from adult tissues. Traditionally, compared to embryonic tissues, adult tissues are thought to express lower levels of telomerase, an enzyme responsible for maintaining telomere length and chromosomal stability (8, 29). Indeed, the majority of adult organs and tissues except for the bone marrow have a limited capacity for self-renewal and cell lineage differentiation. The present studies clearly demonstrate that similar to ESCs, MSCs from adipose tissue express high levels of biologically active telomerase, and they can form colonies and differentiate into cardiovascular cells. These results strongly support the idea that the adipose tissue stroma contains multipotent adult stem cells that may serve as a new resource of adult stem cells for tissue engineering and cellular therapy. The reports by others of the development of cardiac myogenic cells from adult adipose tissue stromal cells (5-7) are based on studies using different cell culture systems and additional stimulation.

The ribonucleoprotein telomerase plays a key role in maintaining telomere length in stem cells and immortal and actively dividing cells (29). Expression of telomerase is developmentally regulated in the heart. Previous work by Borges and Liew (11) has shown that telomerase activity declines rapidly after birth, and become almost undetectable within three weeks of birth. The disappearance of telomerase activity at the time that cardiomyocytes become terminally differentiated suggests that telomerase down-regulation is important in the permanent withdrawal of cardiomyocytes from the cell cycle. It is largely unknown whether cells in a highly differentiated adipose tissue express telomerase. Our current study showed, for the first time, the presence of biologically active telomerase as well as myocardin at high levels in the MSC population of the adipose tissue stroma. The dually positive MSCs show a great potency of self-renewal and myogenic development when cultured in vitro. Thus, resembling embryonic cells, adult MSCs in adipose tissue stroma are a group of undifferentiated, pluripotent stem cells capable of differentiating into multiple cell lineages. However, at the present time, we do not know whether telomerase expression has any direct impact on the development of cardiovascular cell lineages. Further investigation of MSCs with genetically manipulated telomerase may facilitate the clarification of the role for telomerase in regulation of MSC-myocyte differentiation.

Expression of myocardin, an extraordinarily potent transcriptional activator of serum response factor (SRF) (16, 21), may represent a new mechanism that regulates cardiac and smooth muscle development. Myocardin belongs to the SAP (SAF-A/B, Acinus, PIAS) domain family of nuclear proteins that regulate diverse aspects of chromatin remodeling and transcription. In embryonic tissue, myocardin is initially synthesized in the cardiac crescent at the time of cardiogenic specification and is maintained throughout the atrial and ventricular chambers of the heart during later development. Myocardin is also expressed in embryonic vascular smooth muscle cells within the cardiac outflow tract and aortic arch arteries, as well as in developing visceral smooth muscle cells of the respiratory, gastrointestinal, and genitourinary tracts. However, myocardin is neither expressed in the coronary vasculature and dorsal aorta, nor in skeletal muscle cells. Furthermore, derived from alternative splicing of the myocardin gene, myocardin A has been found to be the most abundant isoform in the heart from embryo to adult (17). Our observation that adipose tissue-derived MSCs express myocardin-A points to the possibility that myocardin may play a role in regulation of cardiomyogenic cell maturation from MSCs. The relationship between telomerase and myocardin is very intriguing as they co-exist in stem cells while carrying out different functions: telomerase regulates the cell senescence and myocardin controls myogenesis. Since MSCs positive for both telomerase and myocardin show a greater potency towards cardiac myogenic development, it is likely that the two molecules interact in regulation of MSC growth as well as myogenesis.

Transplantation of adult stem cells with the potency of differentiation or transdifferentiation into cardiovascular myocytes offers a new vehicle to repopulate cardiac cells and repair damaged myocardium. In the studies disclosed herein, we provide evidence that the mesenchymal cell population from adipose tissue contains a stem cell lineage which expresses telomerase, myocardin and cardiomyogenic proteins. The adipose tissue-derived stem cells are capable of transforming into myogenic cells that become mature, beating myocytes. Our observations presented here indicate that pluripotent stem cells, including cardiomyocyte and vascular progenitor cells, exist in adipose tissues. Employing various techniques (e.g., immunocytochemistry, morphological examination, ultrastructural analysis, and electrophysiological assessment), Planat-Benard, et al. (7) demonstrated that mesenchymal stem cells can differentiate into ventricle- and atrial-like cells which also respond to stimulation with adrenergic agonists.

Serving as an alternative stem cell reservoir, adipose tissue has several advantages over other sources of stem cells for cellular therapy: abundance, accessibility, and replenishable source of adult stem cells that can be isolated from liposuction waste tissue by collagenase digestion and differential centrifugation. Although the adipose tissue-derived adult stem cells have been reported to differentiate into the adipocyte, chondrocyte, myocyte, neuronal, and osteoblast lineages (3), in cultures, we found that they mainly develop into myogenic and connective tissue cells primarily seen in wound healing. Although the in vitro data do not reproduce precisely the situation found in a living heart, these data are believed to be indicative of at least some similar effects that will be obtained in vivo. In vivo experiments transplanting the MSCs derived from adipose tissue into the hearts of animals with experimentally created myocardial infarction or ischemia are currently underway in our laboratory. Confirmation of in vivo myogenesis from adipose tissue-derived adult stem cells is expected to lead to clinical application of adipose tissue MSCs in treatment of selected patients with heart disease.

### REFERENCES

The following publications are cited in the foregoing text by numbers enclosed in parentheses.
1. Perin, E. C., Dohmann, H. F., Borojevic, R., Silva, S. A., Sousa, A. L., Mesquita, C. T., Rossi, M. I., Carvalho, A. C., Dutra, H. S., Dohmann, H. J., Silva, G. V., Belem, L., Vivacqua, R., Rangel, F. O., Esporcatte, R., Geng, Y. J., Vaughn, W. K., Assad, J. A., Mesquita, E. T. & Willerson, J. T. (2003) Circulation 107, 2294-302.
2. Smits, P. C., van Geuns, R. J., Poldermans, D., Bountioukos, M., Onderwater, E. E., Lee, C. H., Maat, A. P. & Serruys, P. W. (2003) J Am Coll Cardiol 42, 2063-9.
3. Gimble, J. & Guilak, F. (2003) Cytotherapy 5, 362-9.
4. De Ugarte, D. A., Ashjian, P. H., Elbarbary, A. & Hedrick, M. H. (2003) Ann Plast Surg 50, 215-9.
5. Rangappa, S., Fen, C., Lee, E. H., Bongso, A. & Wei, E. S. (2003) Ann Thorac Surg 75, 775-9.
6. Gaustad, K. G., Boquest, A. C., Anderson, B. E., Gerdes, A. M. & Collas, P. (2004) Biochem Biophys Res Commun 314, 420-7.
7. Planat-Benard, V., Menard, C., Andre, M., Puceat, M., Perez, A., Garcia-Verdugo, J. M., Penicaud, L. & Casteilla, L. (2004) Circ Res 94, 223-9.
8. Harley, C. B. & Villeponteau, B. (1995) Curr Opin Genet Dev 5,249-55.
9. Cech, T. R. & Lingner, J. (1997) Ciba Found Symp 211, 20-8; discussion 28-34.
10. Odorico, J. S., Kaufman, D. S. & Thomson, J. A. (2001) Stem Cells 19, 193-204.
11. Borges, A. & Liew, C. C. (1997) J Mol Cell Cardiol 29, 271-24.
12. Leri, A., Malhotra, A., Liew, C. C., Kajstura, J. & Anversa, P. (2000) J Mol Cell Cardiol 32, 385-90.
13. Leri, A., Barlucchi, L., Limana, F., Deptala, A., Darzynkiewicz, Z., Hintze, T. H., Kajstura, J., Nadal-Ginard, B. & Anversa, P. (2001) Proc Natl Acad Sci U S A 98, 8626-31.
14. Oh, H. & Schneider, M. D. (2002) J Mol Cell Cardiol 34, 717-24.
15. Oh, H., Wang, S. C., Prahash, A., Sano, M., Moravec, C. S., Taffet, G. E., Michael, L. H., Youker, K. A., Entman, M. L. & Schneider, M. D. (2003) Proc Natl Acad Sci U S A 100, 5378-83.
16. Wang, D., Chang, P. S., Wang, Z., Sutherland, L., Richardson, J. A., Small, E., Krieg, P. A. & Olson, E. N. (2001) Cell 105, 851-62.
17. Ueyama, T., Kasahara, H., Ishiwata, T., Nie, Q. & Izumo, S. (2003) Mol Cell Biol 23, 9222-32.
18. Wang, Z., Wang, D. Z., Hockemeyer, D., McAnally, J., Nordheim, A. & Olson, E. N. (2004) Nature 428, 185-9.
19. Wang, Z., Wang, D. Z., Pipes, G. C. & Olson, E. N. (2003) Proc Natl Acad Sci U S A 100, 7129-34.
20. Li, S., Wang, D. Z., Wang, Z., Richardson, J. A. & Olson, E. N. (2003) Proc Natl Acad Sci U S A 100, 9366-70.
21. Wang, D. Z., Li, S., Hockemeyer, D., Sutherland, L., Wang, Z., Schratt, G., Richardson, J. A., Nordheim, A. & Olson, E. N. (2002) Proc Natl Acad Sci USA 99, 14855-60.
22. Anversa, P., Leri, A., Kajstura, J. & Nadal-Ginard, B. (2002) J Mol Cell Cardiol 34, 91-105.
23. Min, J. Y., Yang, Y., Converso, K. L., Liu, L., Huang, Q., Morgan, J. P. & Xiao, Y. F. (2002) J Appl Physiol 92, 288-96.
24. Klug, M. G., Soonpaa, M. H., Koh, G. Y. & Field, L. J. (1996) J Clin Invest 98, 216-24.
25. Muller-Ehmsen, J., Whittaker, P., Kloner, R. A., Dow, J. S., Sakoda, T., Long, T. I., Laird, P. W. & Kedes, L. (2002) J Mol Cell Cardiol 34, 107-16.
26. Kocher, A. A., Schuster, M. D., Szabolcs, M. J., Takuma, S., Burkhoff, D., Wang, J., Homma, S., Edwards, N. M. & Itescu, S. (2001) Nat Med 7, 430-6.
27. Taylor, D. A. (2001) Curr Control Trials Cardiovasc Med 2, 208-210.
28. Asahara, T., Murohara, T., Sullivan, A., Silver, M., van der Zee, R., Li, T., Witzenbichler, B., Schatteman, G. & Isner, J. M. (1997) Science 275, 964-7.
29. Tzukerman, M., Selig, S. & Skorecki, K. (2002) J Pediatr Endocrinol Metab 15, 229-40.

Without further elaboration, it is believed that one skilled in the art can, using the description herein, utilize the present invention to its fullest extent. The foregoing embodiments are to be construed as illustrative, and not as constraining the remainder of the disclosure in any way whatsoever. While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims. The disclosures of all patents, patent applications and publications cited herein are hereby incorporated herein by reference, to the extent that they provide exemplary, procedural or other details supplementary to those set forth herein.

What is claimed is:

1. An in vitro method of producing cardiovascular myocytes comprising:
(a) isolating myogenic stem cells from the stromal or mesenchymal compartment of adult mammalian adipose tissue;
(b) culturing the isolated cells in a medium that favors myogenic development of said stem cells; and
(c) harvesting cardiovascular myocytes from said culture medium.

2. The method of claim 1 comprising (d) testing a sample of cells for production of telomerase.

3. The method of claim 1 comprising (e) testing a sample of cells for production of myocardin.

4. The method of claim 1 comprising (f) testing a sample of cells for production of at least one cardiomyogenic protein.

5. The method of claim 1 wherein step (a) comprises
(a-1) transfecting the resulting isolated myogenic stem cells with cDNA encoding telomerase and myocardin;
step (b) comprises:
(b-1) culturing the resulting transfected cells in a medium that favors myogenic development of said stem cells;
(b-2) expressing the transfected telomerase and myocardin cDNA; and
(b-3) expressing at least one other gene in said transfected myogenic stem cells encoding at least one protein associated with telomerase and myocardin function.

6. The method of claim 1 wherein said culturing comprises:
(b-4) plating stromal or mesenchymal cells at a density of about 10,000 cells/cm² in an initial cell culture medium comprising DMEM:F12 medium supplemented with penicillin (100 U/mL), streptomycin sulfate (100 μg/mL) and 10% fetal bovine serum (FBS);
(b-5) replacing said initial medium with an inducing medium comprising Iscove's MDM liquid medium (Gibco, Carlsbad Calif.) supplemented with L-glutamine (2 mmol/L), penicillin (100 U/mL), streptomycin sulfate (100 μg/nl), 0.1 mM nonessential amino acid, 10⁴ mol/L 2-mercaptoethanol and 20% FBS, to induce cardiomyogenesis; and
(b-6) allowing cardiovascular myocytes to grow until confluence is reached.

7. A composition for treating a cardiovascular disorder comprising:
a plurality of cardiovascular myocytes prepared by the method of any of claims 1-6; and
a pharmaceutically acceptable carrier.

8. A method of treating a mammalian subject suffering from a cardiovascular disorder, the method comprising:
a) providing a plurality of myogenic stem cells obtained from the stromal or mesenchymal compartment of adult adipose tissue;
b) causing said stem cells to proliferate;
c) inducing differentiation of said stem cells into cardiovascular myocytes;
d) implanting at a cardiovascular site in said subject a composition comprising said stem cells and/or said differentiated cardiovascular myocytes.

9. The method of claim 8 wherein said step d) comprises providing muscle cells in the heart of the subject.

10. The method of claim 8 wherein said step d) comprises providing muscle cells in a blood vessels of the subject.

11. The method of claim 8 further comprising c′) proliferating and isolating said myogenic stem cells in vitro prior to said implanting.

12. The method of claim 8 wherein said implanted myogenic stem cells produce telomerase and myocardin at the implantation site.

13. The method of claim 12 wherein said telomerase production deters stem cell senescence and apotosis.

14. The method of claim 12 wherein said differentiation is promoted by said telomerase and myocardin.

15. The method of claim 12 wherein said stem cells have been engineered to co-express telomerase and myocardin.

16. The method of claim 8 wherein step a) comprises obtaining adipocytes by:
(a-i) liposuctioning adipose tissue from the stromal or mesenchymal compartment of the body of said subject or from that of a donor, to provide a quantity of removed adipose tissue;
(a-ii) enzymatically digesting proteins and DNA in said removed adipose tissue, to provide a quantity of digested adipose tissue; and
(a-iii) separating live adipocytes from other cells in said digested tissue.

17. The method of claim 8 comprising identifying, selecting and growing cardiovascular stem cells in vitro.

18. The method of claim 8 comprising identifying, culturing and selecting cardiac myogenic cells and/or heart muscle cell progenitors.

19. The method of claim 8 comprising identifying, culturing and selecting vascular myogenic cells and/or vascular smooth muscle cell progenitors.

20. The method of claim 8 comprising identifying, culturing and selecting endothelial cell progenitors.

21. The method of any of claims 18-20 wherein said step of selecting comprises a cell sorting technique.

22. The method of claim 8 comprising repopulating functional cardiovascular myocytes and endothelial cells to repair damaged myocardium at said site.

23. The method of claim 8 wherein said site comprises a myocardial infarction.

24. The method of claim 8 wherein said disorder comprises chronic heart failure.

25. The method of claim 8 wherein said site comprises a vascular defect, atherosclerosis or hypertension.

26. The method of claim 8 comprising repopulating vascular cells at said site.

27. The method of claim 8 comprising isolating and transplanting stem cells with potential to differentiate into smooth muscle cells.

28. The method of claim 8 comprising isolating and transplanting endothelial cells.
